(19) United States
(12) Patent Application Publication
(10) Pub. No.: US 2010/0049191 A1
(43) Pub. Date: Feb. 25, 2010

(54) TISSUE ABLATOR

(75) Inventors: Nagy Habib, London (UK); Andrew Pacey, Hertfordshire (UK)

Correspondence Address: OPPENHEIMER WOLFF & DONNELLY LLP, 45 SOUTH SEVENTH STREET, SUITE 3300, MINNEAPOLIS, MN, 55402, UNITED STATES, 612-607-7000, 612-607-7100

(73) Assignee: EMCISION LIMITED, London (UK)

(21) Appl. No.: 12/374,627

(22) PCT Filed: Jul. 23, 2007
(86) PCT No.: PCT/GB2007/02793

§ 371 Date: Sep. 30, 2009

(30) Foreign Application Priority Data
Jul. 21, 2006 (UK) 0614557.7

(51) Int. Cl. A61B 18/14; A61B 1/00

(52) U.S. Cl. 606/41; 600/104

(57) ABSTRACT

A flexible RF device (1) can be deployed through a flexible endoscope. An electrode structure has a central electrode (12) and outer electrode (11). Flexible electrodes (30), circular electrodes (51, 53) and circular loop assemblies (55, 56) with different diameters are also disclosed, as well a tweezer electrodes (41) with pads (43) for increasing contact area. Retractable electrodes (100) are also disclosed.

The present invention relates to an electromagnetic energy delivery device and method and to electrodes for such device.

This invention is in the field of tumour treatment using heat. It is well known that heating tissue, or tissue ablation will cause cell death and this can be used to kill tumours in-situ. Heat can also be used to cauterize vessels and stop bleeding. The heat can be applied using RF current, microwave, or ultrasound radiation. The heating energy can be applied directly to the tissue, these can be delivered directly to the organ in question, or via a laparoscopic port, or endoscopically.

### BRIEF DESCRIPTION OF THE PRIOR ART

U.S. Pat. Nos. 5,976,129 and 5,662,680 (Desai) describe an endoscopic device for RF coagulation of uterine fibroids using bipolar or monopolar RF energy and the object of the invention is to provide a device with control means for continuous irrigation and evacuation of a body cavity. However, the endoscopic device has a straight access conduit. Electrodes are enclosed with sheeths which have bendable portions, bendable by the surgeon pulling on guide wires. The device has limited application and limited electrode configurations. U.S. Pat. No. 6,918,906 (Long) describes an endoscopic ablation device which is fitted to the terminal end of an endoscope with electrode wires affixed to the outside of the endoscope. The wires may contact the patient, which is not ideal, and the device only appears suitable for use with a limited range of endoscopes.

U.S. Pat. No. 6,530,922 (Cosman) describes multiple electrodes which cause reduced tissue damage, which may also be mounted on a carrier, but does not describe a carrier which can itself be an electrode. Similarly, US 22120260, US 22120261 and US 25137662 (Morris) describe multiple electrodes mounted on a carrier, but also does not describe a carrier which can itself be an electrode. Although endoscopic devices are described, they are relatively complicated and suitable only for needle-type electrodes.

The present invention aims to alleviate at least to a certain extent the problems of the prior art.

### SUMMARY OF INVENTION

Various aspects of the invention are set out in the independent claims. Various optional features are set out in the dependent claims.

Another aspect of the invention provides a flexible device that can be delivered through the channel of a standard endoscope and can apply RF energy to tissue on the inner wall of the stomach or other parts of the digestive tract, the lungs, the prostate, the urinary tract, or the uterus. The device is also suitable for patients with portal hypertension who have oesophageal and gastric varices which can bleed. RF application on both sides of the vessels can thrombose the vascular channel. The device may further be used as prophylaxis to prevent bleeding or can be applied in an emergency to stop bleeding. An example would be use in the rectum to thrombose piles in patients with anal haemorrhoids.

The energy, e.g. RF energy, may be applied in a monopolar or more preferably a bipolar manner in any of the aspects of the invention, and can either be used to ablate a tumour on the stomach wall or to seal blood vessels to prevent bleeding. In preferred embodiments, the device may use the end face of the device as one electrode in a ring and needle configuration and/or flexible tape configurations to deliver RF energy in a controlled manner from a variety of contact angles and to ablate to a selectable and determined depth. Bipolar application ensures a high degree of controllability, which can be controlled in depth by using the end face of the device as an electrode of opposite polarity to the needles.

### BRIEF DESCRIPTION OF FIGURES

The present invention may be carried out in various ways and various preferred embodiments of devices and methods in accordance with the invention will now be described by way of example only with reference to the accompanying drawings, in which:

FIG. 1 shows the application of the device to the target site;

FIG. 2 shows an embodiment of the device;

FIG. 3 shows detail of the distal end of the device;

FIG. 4 shows an alternate embodiment of the distal end of the device;

FIG. 5 shows another alternate embodiment of the distal end of the device;

FIG. 6 shows another alternate embodiment of the distal end of the device;

FIG. 7 shows another alternate embodiment of the distal end of the device;

FIG. 8 shows detail of the distal end of the device depicted in FIG. 7;

FIG. 9 shows another alternate embodiment of the distal end of the device.

FIGS. 10 and 11 shows modifications of the FIG. 9 embodiment; and

FIG. 12 shows a test matrix used with the device of FIG. 3.

### DETAILED DESCRIPTION OF THE INVENTION

The device uses RF power to heat the tissue in the frequency range 200 kHz to 800 kHz, typically at 450 kHz, and is a bipolar device, so the RF current is applied between two electrodes applied to the target site, the two electrodes are connected to opposite polarities of an RF generator.

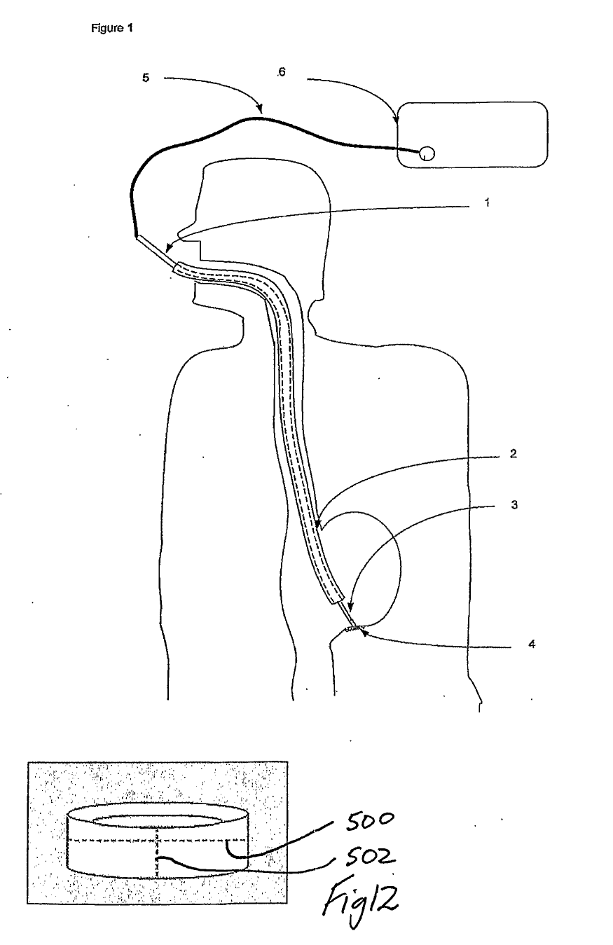

FIG. 1 shows the application of the device. The device 1, is inserted through the channel of an endoscope 2. At the distal end of the device an electrode assembly 3 makes contact with the treatment area 4 which is on the wall of the stomach or other part of the digestive system. At the proximal end a cable 5 is connected to a RF generator 6.

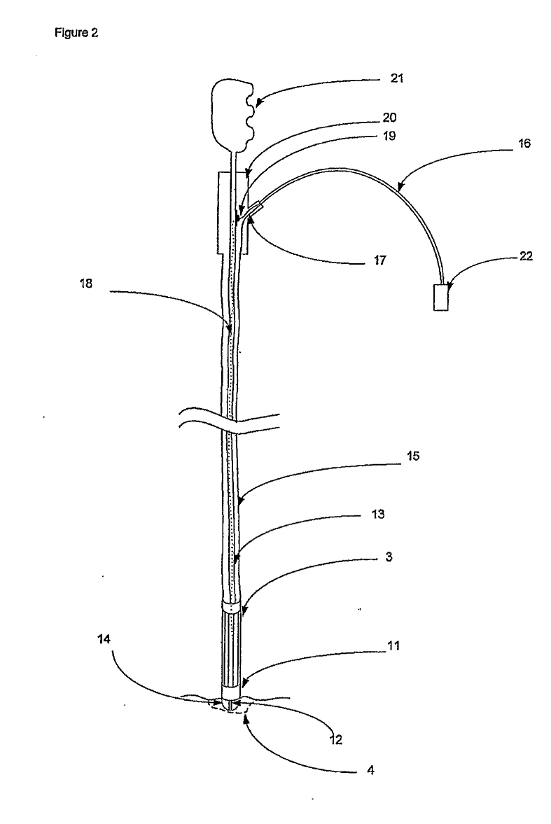

More detail on the device is given in FIG. 2. The electrode assembly 3 consists of an outer electrode 11, and a central electrode assembly 12. The outer electrode is bonded to an outer tube of the device 15, which may be a flexible polymer such as polyethylene. An electrical connection to the outer electrode is made with a wire 17, the wire may be embedded in the wall of the outer tube, or mounted in a channel within the wall of the outer tube.

The central electrode is connected to a central tube 13, which can slide within the main body of the device to extend and withdraw the central electrode. The central electrode is connected to a wire 18, which is mounted inside the central tube. When deployed the outer electrode will make contact with the surface of the treatment area 4. The outer electrode may have micro-needles mounted on to penetrate the tissue up to 1 mm. The central electrode 12 can be pushed into the tissue a distance of between 1 and 50 mm, typically to a maximum of 6 mm. The heated volume will be a hemispherical volume 14. The whole of the treatment volume 4 can be ablated by successive applications of the device.

The device is typically over 1 metre long, sufficient to protrude from the channel of an endoscope. At the proximal end the outer electrode wire is connected to one conductor of a multi-core cable 16, the wire may be embedded in the wall of the outer tube. The outer tube is bonded to a Y-connector 20, the Y-connector houses a lumen though which the central tube passes, permitting movement of the central tube. The other conductor of the multi-core connector is connected to the central needle wire via a slidable contact 19. One end of the cable 16 is connected to a plug 22, and the other end is attached to the Y-connector. The proximal end of the central tube is attached to a handle 21 to aid deployment of the central tube and with it the central needle.

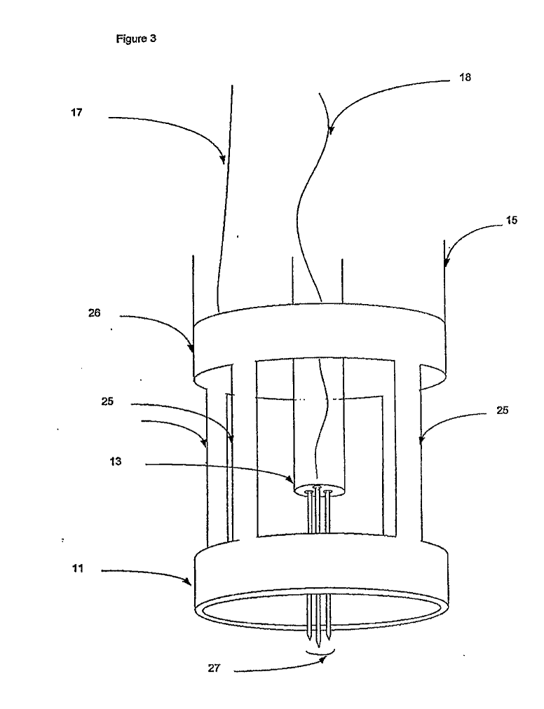

Further details of the electrode assembly is given in FIG. 3. The outer-electrode 11 is attached to the outer body 15 via struts 25. The apertures between the struts permit visualization of the distal electrodes by the endoscope optics. The struts are made of conductive material such as stainless steel but they may have an insulated coating of a polymer such as parylene (Specialty Coatings Ltd). The proximal end of the outer electrode 26 is attached to the outer tube 15, and connected to the wire. The central electrode is shown in an embodiment with 3 micro-needles 27, attached to the central tube 13 and electrically connected to a wire 18. The central electrode carrier 13 may be larger in diameter and may make insulated contact with the outer electrode 11 which may act to limit the depth of needle travel.

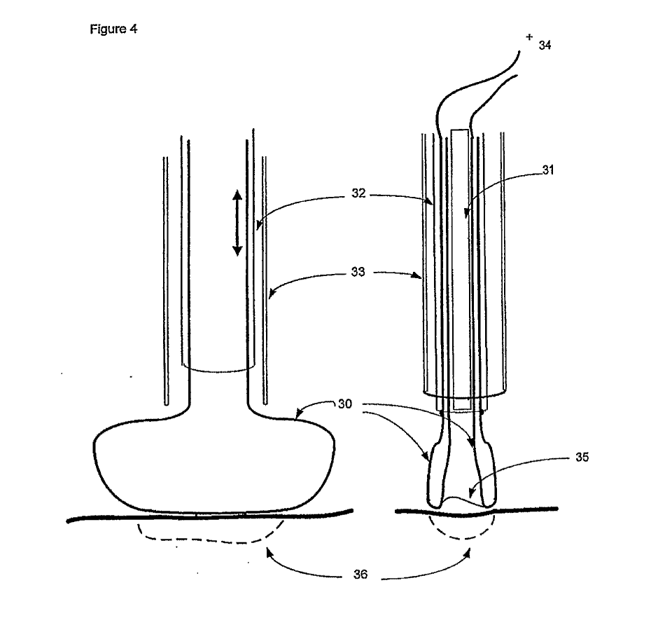

Another embodiment is shown in FIG. 4. There are two flexible electrodes 30 attached to the central tube, and no outer electrode. The flexible electrodes consist of loops of a conducting wire or strip. The two loops are separated by a spacer 31, and are deployed by pushing out the central tube 32. When deployed the loops will flatten on the tissue surface to form two line electrodes. Flexible non-conducting spacers 35 connect the loops to prevent them splaying out and to maintain the correct separation. Each loop is connected to one polarity of an RF generator in bipolar mode 34, so that the strip of tissue between the two electrodes is heated. Before and after deployment the loops are withdrawn into the outer body 33 by retracting the central tube 32, permitting the device to be inserted through the endoscope channel. The conducting loops 30 can be fabricated from a superelastic material such as nitinol or an elastic material such as stainless steel. The flexible spacer 35 can be nylon cord. In an alternate implementation the conductors can be tracks on a flexible PCB, such as gold tracks on polyimide, in this case there will be a single hoop with two conductors mounted on it.

This embodiment has the advantage over that in FIG. 2 in that the treated area 36 is an elliptical strip that is longer than the diameter of the outer tube. The treated area will be shallow as the electrodes do not penetrate the tissue, so this embodiment is suitable for large area shallow target areas.

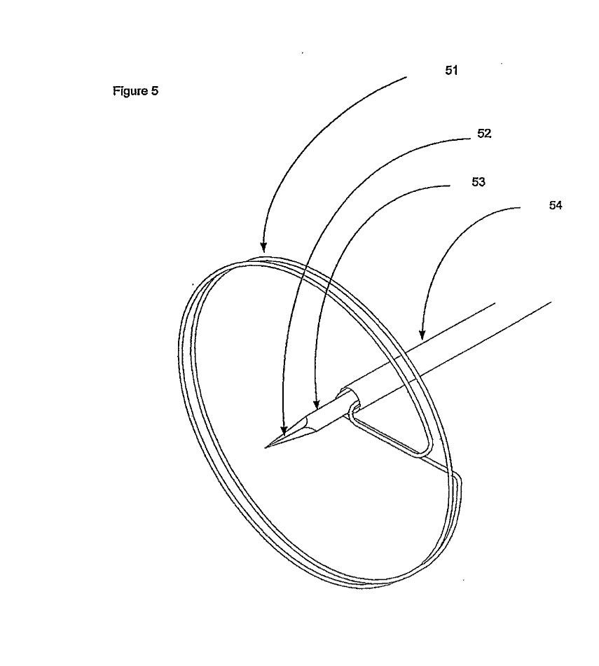

Another embodiment using a flexible electrode is shown in FIG. 5. The outer electrode 51 is fabricated from a wire made from a superelastic material such as nitinol or an elastic material such as stainless steel. When pushed out of the outer body it is preformed to adopt the shape of a loop of a fixed diameter, and will lie on the tissue surface to form a circle. The loop may have one or more turns. This electrode is connected to one polarity of an RF generator. The central electrode is made of one or more needles 53, the tip of the needle 52 is exposed to permit electrical contact. The body of the needle 53 is insulated using a heat shrink material such as Teflon, to prevent shorting to the outer loop. The central electrode is connected to the opposite polarity of the REF generator. When power is applied across the two electrodes, the circular region circumscribed by the outer circle will be heated. When the outer electrode is retracted it will fold into the outer body in a spiral form.

In another embodiment shown in FIG. 6 there are two circular loop assemblies 55, 56, with different diameters. The two loop assemblies are connected to opposite polarities of an RF generator, to heat the annular ring between the two loops. A central electrode can be used with the two loops, and when the central electrode is deployed it will be connected to one polarity of the RF generator, and the inner loop is connected to the opposite polarity.

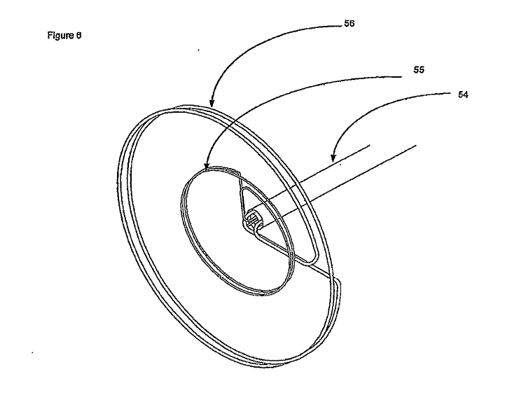

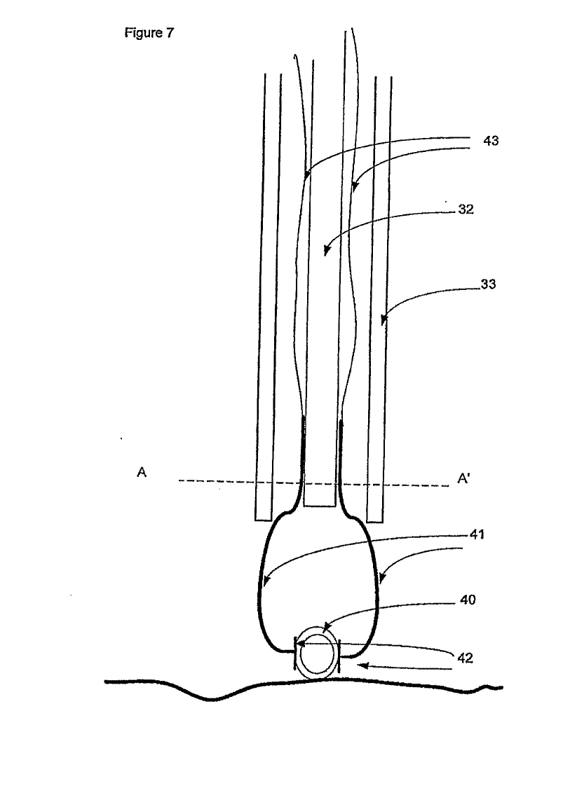

Another embodiment is shown in FIG. 7, this embodiment can be used to heat a target area such as a blood vessel 40. Two electrodes 41 are arranged as tweezers, and connected to opposite polarities of an RF generator using wires 43. The electrodes are attached to the central tube 32, and when this is retracted will fold inside the outer tube 33. The electrodes are deployed by pushing the central tube which will open up the electrodes, and clamped around the outside of the blood vessel by pulling the central tube back so the electrode tips are forced together by the outer tube. The electrodes can be fabricated from a super-elastic material such as nitinol, and can be pre-set into the shape shown. The electrode tips may have pads 43 to increase the contact area on the vessel wall. This embodiment can be used to seal blood vessels, such as those in gastric varices, oesophageal varices, and haemorrhoids.

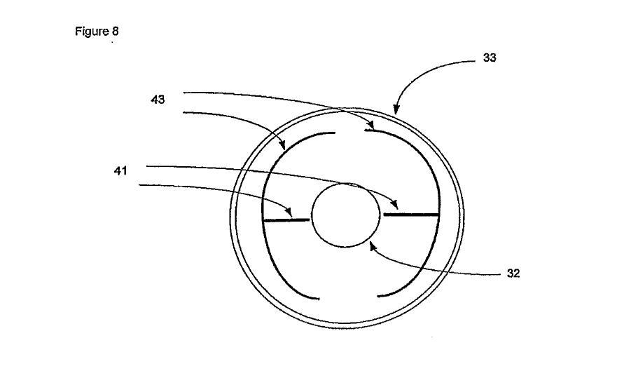

Details of one configuration of the electrode tips are shown in FIG. 8 which corresponds to section A-A′ of FIG. 7, with the electrodes retracted inside the tube. The tips 43 are constructed of rectangular sheets of a conductive and elastic material such as nitinol or stainless steel. They are formed in a semi-circular pattern that can be stowed inside the outer tube 33. When clamped around the vessel, the force of the clamping will flatten the electrode tips along the vessel, and this will permit a greater length of the vessel to be heated. This will permit the coagulation of a larger diameter vessel.

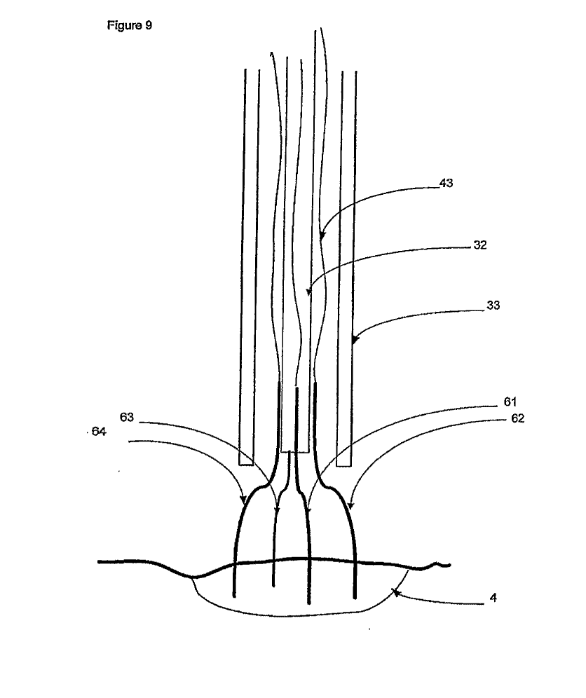

FIG. 9 shows another embodiment where the electrodes are flexible needles 61,62,63,64. These needles are fabricated from an elastic material such as stainless steel, or a superelastic material such as nitinol, and connected to wires 43. The needles when withdrawn will fold inside the outer body 33. When deployed the central tube 32 is pushed forward relative to the outer tube, pushing the needles forward, and they will adopt a preformed shape and splay out, so that the needles lie on a diameter that is greater than the diameter of the outer tube. The needles are inserted into a treatment region 4. Two or more needles are used, and connected to opposite polarities of an RF generator. In the embodiment shown 4 needles are deployed, and needles 61 and 63 are connected to the same polarity of an RF generator, and 62, 64, connected to the opposite polarity. This will supply current to the perimeter of a circle defined by the needles, and heat a cylinder defined by this circle with a depth determined by the depth of the needles in the tissue. The diameter of the total cylindrical volume heated will be larger than the diameter of the outer tube. Other numbers and configurations of needles are possible.

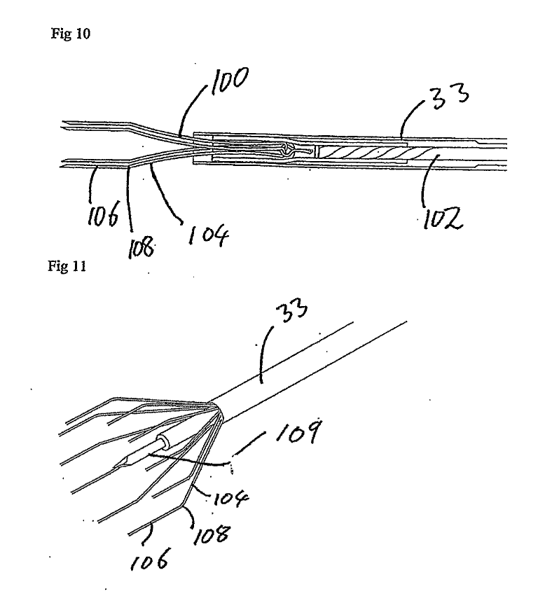

FIGS. 10 and 11 show modifications of the embodiment of FIG. 9. In FIG. 10, retractable electrodes 100 are sprung and moveable by steel flexible shaft 102. Electrodes are each made up of substantially straight first 104 and second 106 portions with a kink 108 therebetween, the needle electrodes 100 therefore having little or no curvature. FIG. 11 shows a similar arrangement but with ten needles instead of four and with a retractable central electrode 109 which may be fully or partially retracted into tube 33 from the position shown, as desired by the surgeon/operator.

All of the embodiments of devices described may be deployed through the full length of standard endoscope channels, being insertable through a proximal end thereof and slideable all of the way therethrough for deployment at or out of a distal end thereof as shown in FIG. 1.

For the validation of the device shown in FIG. 3, fresh bovine liver (not shown) was used with a text matrix shown in FIG. 12 in which 500 is diameter and 502 is depth. A Rita Medical RF generator (Model 1500) (not shown) was used to generate the power. The device of FIG. 3 was connected to the generator via an adaptor cable.

The device was placed on the surface of the bovine liver; the generator was set at 1 Watt and the power was applied. The timer was started in order to record the time taken for the impedance reading to increase by 10% over baseline, which should be sufficient to induce tissue coagulation. The generator was then put in standby mode. The coagulated tissue was resected and measured.

The device was relocated and the process was repeated a total of ten times.

The results are described below in Table 1.

TABLE 1
Test Results

| | Watts | Impedance | RF Time | | |
|---|---|---|---|---|---|
| | Delivered | (starting) | in mins | Diameter | Depth |
| Example 1 | 1 | 630 | 0.1 | 1.78 | 1.80 |
| Example 2 | 1 | 563 | 0.2 | 2.45 | 1.90 |
| Example 3 | 1 | 485 | 0.2 | 2.89 | 1.76 |
| Example 4 | 1 | 365 | 0.1 | 2.90 | 1.60 |
| Example 5 | 1 | 470 | 0.1 | 2.57 | 1.85 |
| Example 6 | 1 | 553 | 0.2 | 2.98 | 2.13 |
| Example 7 | 1 | 641 | 0.2 | 3.28 | 2.03 |
| Example 8 | 1 | 413 | 0.3 | 2.71 | 2.89 |
| Example 9 | 1 | 504 | 0.2 | 3.12 | 1.98 |
| Example 10 | 1 | 378 | 0.1 | 2.13 | 2.03 |

Accordingly, relatively consistent and effective coagulation was shown.

Various modifications may be made to the embodiments described without departing from the spirit and scope of the accompanying claims as interpreted under patent law.

What is claimed is:

1. An electromagnetic energy delivery device which is deployable through an elongate channel extending along a flexible endoscope for delivering electromagnetic energy to tissue, the device having an elongate main body and an electrode assembly at a distal end thereof, the main body being flexible along the length thereof to enable the device to conform to the shape of a channel of a flexible endoscope.

2. A device as claimed in claim 1 in which the main body comprises a tube.

3. A device as claimed in claim 2 in which the electrode is attached to an electrode deployment device which is slideable along inside the tube.

4. An electromagnetic energy delivery device which is deployable through an elongate channel of an endoscope for delivering electromagnetic energy to tissue, the device having an elongate main body and an electrode assembly at a distal end thereof, the electrode assembly including a non-penetrating electrode arranged to lie against tissue to provide electromagnetic energy thereto.

5. A device as claimed in claim 4 in which the non-penetrating electrode comprises a ring.

6. A device as claimed in claim 5 in which the ring has an outer diameter substantially equal to an outer diameter of the elongate main body.

7. A device as claimed in claim 5 in which the non-penetrating electrode has a first annular part secured to the distal end of the main body and a second annular part spaced by a plurality of struts from the first annular part.

8. A device as claimed in claim 4 in which the electrode assembly includes a central electrode assembly located coaxially with the ring electrode, the central electrode assembly preferably having at least one needle electrode.

9. A device as claim 4 in which the non-penetrating electrode comprises at least one loop element.

10. A device as claimed in claim 9 in which the loop element is flexibly expandable to a cross-dimension larger than the cross-dimension of the main body of the device, the loop being retractable at least partly into the main body.

11. A device as claimed in claim 9 in which two said loop elements are provided, the loop elements preferably being spaceable apart by a flexible spacer.

12. A device as claimed in claim 4 in which the non-penetrating electrode comprises a wire hoop, the hoop having one or more turns.

13. A device as claimed in claim 12 in which the hoop is foldable for retraction into the main body.

14. A device as claimed in claim 12 or claim 13 which includes two said hoops of different diameters.

15. A device as claimed in claim 4 in which the non-penetrating electrode comprises a contact pad adapted to be placed next to a vessel to be treated.

16. A device as claimed in claim 4 in which the main body is flexible for conforming to the shape of a channel extending along a flexible endoscope.

17. A device as claimed in claim 4, in which the main body is tubular and has a proximal end, at least one power line extending along the main body from the proximal end to the distal end thereof.

18. A device as claimed in claim 1 in which the electrode assembly is arranged to supply monopolar or bipolar radio frequency energy to tissue.

19. A device as claimed in claim 1 which is expandable from a first configuration to an expanded use configuration.

20. A device as claimed in claim 1, which includes an electrode assembly which is expandable from a first configuration to an expanded use configuration, the electrode assembly being stored at least partly inside the main body when in the first configuration.

21. A device as claimed in claim 20 in which the main body comprises a tube into which the electrode assembly is at least partly retractable from the use configuration.

22. A device as claimed in claim 20 in which the electrode assembly is mounted upon a deployment member which is slidable in said main body for expanding or retracting the electrode assembly; the deployment member preferably comprising a tube.

23. A device as claimed in claim 20 in which the electrode assembly includes at least one expandable flexible ring electrode; or at least one expandable electrode having a sheet-like form.

24. A device as claimed in claim 20 in which the electrode assembly includes at least one flexible strip electrode.

25. A device as claimed in anyone of claims 20 in which the electrode assembly comprises a plurality of expandable flexible needle electrodes.

26. An electromagnetic energy delivery electrode assembly for applying energy to tissue, electrode assembly including a ring-shaped electrode.

27. An assembly as claimed in claim 26 in which the electrode assembly includes a support for the ring electrode which defines at least one observation window for endoscopic viewing in the region of the ring-shaped electrode.

28. An assembly as claimed in claim 27 in which the support comprises a plurality of mutually spaced struts.

29. An assembly as claimed in claimed 26 in which the ring electrode is flexibly expandable and arranged for deployment from a tubular structure to an expanded deployed configuration in which the ring electrode has a cross dimension larger than that of the tubular structure.

30. An electrode assembly for providing electromagnetic energy to tissue, the assembly having electrodes arranged to clamp around tissue or a vessel to provide electromagnetic energy thereto.

31. An assembly as claimed in claim 28 in which each electrode is sheet-like in form, preferably being a part-cylindrical shape.

32. A method of performing endoscopic surgery which comprises inserting an endoscope into a patient, deploying a device as claimed in any preceding claim longitudinally through a channel of the endoscope, and applying electromagnetic energy to tissue of the patient using the device.

33. An endoscopic surgery apparatus comprising an endoscope having a deployment channel extending longitudinally therethrough, and a device as claimed in claim 1, the device being deployable through and along the channel for performing endoscopic electromagnetic energy delivery surgery on tissue inside a patient.

34. An endoscopic surgery apparatus as claimed in claim 33 which is flexible.

35. An endoscopic surgery apparatus comprising an endoscope having a deployment channel extending longitudinally therethrough, and a device as claimed in claim 5, the device being deployable through and along the channel for performing endoscopic electromagnetic energy delivery surgery on tissue inside a patient.

36. A device as claimed in claim 3 in which the deployment device comprises a tube.

37. A device as claimed in claim 9 in which said loop element comprises an element selected from the group of a wire element and a strip element.

38. A device as claimed in claim 4 in which the electrode assembly is arranged to supply monopolar or bipolar radio frequency energy to tissue.

39. A device as claimed in claim 4 which is expandable from a first configuration to a second expanded use configuration.

40. A device as claimed in claim 4, which includes an electrode assembly which is expandable from a first configuration to a second expanded use configuration, the electrode assembly being stored at least partly inside the main body when in the first configuration.

41. A device as claimed in claim 40 in which the main body comprises a tube into which the electrode assembly is at least partly retractable from the use configuration.

42. A device as claimed in claim 40 in which the electrode assembly is mounted upon a deployment member which is slidable in said main body for expanding or retracting the electrode assembly; the deployment member preferably comprising a tube.

43. A device as claimed in claim 40 in which the electrode assembly includes at least one expandable flexible ring electrode; or at least one expandable electrode having a sheet-like form.

44. A device as claimed in claim 40 in which the electrode assembly includes at least one flexible strip electrode.

45. A device as claimed in claim 40 in which the electrode assembly comprises a plurality of expandable flexible needle electrodes.

46. An endoscopic surgery apparatus comprising an endoscope having a deployment channel extending longitudinally therethrough, and a device as claimed in claim 4, the device being deployable through and along the channel for performing endoscopic electromagnetic energy delivery surgery on tissue inside a patient.

47. An endoscopic surgery apparatus comprising an endoscope having a deployment channel extending longitudinally therethrough, and a device as claimed in claim 30, the device being deployable through and along the channel for performing endoscopic electromagnetic energy delivery surgery on tissue inside a patient.

48. An endoscopic surgery apparatus as claimed in claim 46 which is flexible.

49. An endoscopic surgery apparatus as claimed in claim 35 which is flexible.

50. An endoscopic surgery apparatus as claimed in claim 47 which is flexible.
